(19) United States
(12) Patent Application Publication
(10) Pub. No.: US 2011/0123977 A1
(43) Pub. Date: May 26, 2011

(54) METHOD FOR TAKING A PLURALITY OF SAMPLES

(75) Inventors: Adrianus Joseph Sprenkels, Lelystad (NL); Johan Gerrit Bomer, Haaksbergen (NL); Colin John Ingham, Wageningen (NL); Johannes Epeüs Theodoor Van Hylckama Vlieg, Marly Le Roi (FR); Willem Meindert De Vos, Ede (NL); Albert Van Den Berg, Nijverdal (NL)

(73) Assignee: DSM IP Assets B.V., Heerlen (NL)

(21) Appl. No.: 12/808,615

(22) PCT Filed: Dec. 19, 2008
(86) PCT No.: PCT/NL2008/050832

§ 371 Date: Oct. 1, 2010

(30) Foreign Application Priority Data
Dec. 19, 2007 (EP) 07123576.6

(51) Int. Cl. C12Q 1/70; C12Q 1/02; C12M 1/34; G02B 21/06

(52) U.S. Cl. 435/5; 435/29; 435/288.7; 359/385

(57) ABSTRACT

The invention provides a method for taking a plurality of samples (200) from a substrate (40) with sample material (20) by using an optical microscope (1000) and a stamp (50). At least part of the stamp (50) is transparent and the stamp (50) is arranged in front of the objective (41). The elevations (58) of the stamp (50) are contacted with sample material (20) in a substrate (40). The samples (200) may be replicated to other substrates.

### FIELD OF THE INVENTION

The invention relates to a method for taking a plurality of samples from a substrate with sample material. The invention also relates to a stamp comprising an array of elevations, which stamp can be used for taking a plurality of samples from a substrate with sample material. The invention further relates to an optical microscope for use in the method of the invention.

### BACKGROUND OF THE INVENTION

Methods for manipulating samples while monitoring the sample and/or the manipulation through a microscope are known in the art.

U.S. Pat. No. 6,411,433 describes for instance a micromanipulator for manipulating a sample on a sample-supporting member in a view range of a microscope, wherein the microscope includes an objective lens and a condenser lens, and wherein the sample supporting member is disposed between the objective lens and the condenser lens. The micromanipulator according to U.S. Pat. No. 6,411,433 comprises a high-stiffness manipulation probe. The micromanipulator further comprises a probe supporting member which has a surface that supports the manipulation probe, wherein the probe supporting member comprises a flat plate of transparent material and which is disposed between the sample supporting member and one of the objective lens and the condenser lens.

U.S. Pat. No. 4,270,838 discloses a micromanipulator comprises a miniature operating tool such as a needle, micro-pipette, micro-electrode or the like, which is disposed on a movable member of a microscope, such as an illuminating condenser lens, which is movable in the direction of the optical axis of an objective lens.

WO2006112713 relates to the field of microbiology. Provided is a method which is particularly powerful for High Throughput Screening (HTS) purposes. More specific a high throughput method for determining heterogeneity or interactions of microorganisms is described.

WO2004025273 relates to a device and a method for carrying out in an optimized and automated manner immunological marking techniques for thin-sectioned tissue. A support plate which can be automatically moved by means of a computer-controlled conveying device and on which several thin sections of tissue are placed on small metal nets is immersed like a die into a liquid that is composed of a washing and marking solution and is placed in several recesses within an object support. Said object support can also be automatically moved.

U.S. Pat. No. 6,753,970 describes a transducer in an imaging optical system for generating optical contrasts in the near-field representation of topographies of an object by outcoupling evanescent waves from the underside of the transducer. The transducer comprises a substrate having a transparent plane-parallel protuberance corresponding to the field size of the imaging optical system and pointing toward the object. The specimen outcouples evanescent waves from an underside of the transducer, where the transducer underside is arranged in a focal plane of the imaging optical system.

WO0038838 describes methods for treating biological samples on microscope slides are set forth. One aspect of is the use of predried reagents in wells on trays onto which the slides are placed, especially the use of predried reagents which dissolve sequentially. Yet another aspect is the use of external controls placed directly on a microscope slide in conjunction with a biological sample to be assayed. The external controls can be conveniently placed on a membrane which can be affixed to the slide. A further aspect is a specially designed tray to allow whole chromosome painting of all chromosomes of a cell sample on a single slide. Preferably a reagent is predried in the well. A further aspect is a method of reacting samples on slides by placing them into a reaction chamber together with a coverslip which has a predried reagent on it.

### SUMMARY OF THE INVENTION

Prior art microscopes do not easily allow taking a plurality of samples. Hence, it is an aspect of the invention to provide an alternative method for taking a plurality of samples from a substrate with sample material, which preferably alleviates the disadvantage(s) of the prior art. In a first aspect, the invention provides a method for taking a plurality of samples from a substrate with sample material by using an optical microscope and a stamp, wherein

the optical microscope comprises an objective with a lens, and a substrate holder, wherein a lens-holder distance between the lens and the substrate holder is variable, and

the stamp comprises at a side a plurality of elevations and wherein at least part of the stamp is transparent,

wherein the method comprises:

arranging at least part of the transparent part of the stamp in front of the objective, with the plurality of elevations directed to the substrate holder;

arranging the substrate with sample material to the substrate holder; and

contacting the elevations with the sample material, thereby providing the plurality of elevations with samples, and wherein contacting the elevations with the sample material is preferably controlled by inspection through the optical microscope; and

preferably subjecting the samples to a process selected from the group consisting of analysis and replication.

In a specific embodiment, the sample material is selected from the group consisting of bacteria, virus, fungi, yeast and any other living cell or tissue.

In an embodiment, contacting the elevations with the sample material is controlled by inspection through the optical microscope. This is especially enabled due to the fact that the stamp is at least partially transparent and arranged in front of the objective. In an embodiment, the stamp substantially consists of a transparent material selected from the group consisting of PMA (polymethacrylate), PMMA (polymethylmethacrylate), PC (polycarbonate), PDMS (polydimethyl siloxane), perspex, quartz, gemstones, glass, ceramic materials (such as transparent ceramics). In an embodiment, the stamp comprises two or more materials. In a specific embodiment, the stamp comprises a base part, comprising a first material, especially a transparent material, such as glass, and an elevation part, comprising the elevations, and comprising a second material, such as PDMS. Since the stamp is at least partially transparent and arranged in front of the objective, visual inspection through the microscope through the transparent part of the stamp is possible, i.e. it is possible to inspect the substrate through transparent part of the stamp and the microscope (i.e. especially the objective).

The stamp may in an embodiment comprise a n*m stamp elevations array of elevations, wherein n and m are independently in the range of 1-10,000, such as 2-10,000, especially 2-5000, wherein neighbouring elevations are arranged at a shortest elevation distance in the range of 1-1000 μm, wherein the elevation height of the elevations is in the range of 0.1-1000 μm. The optical microscope is especially a microscope for visible light. However, the optical microscope may in an embodiment also especially be a microscope for IR or UV light. In a specific embodiment of the substrate, the substrate comprises a porous substrate such as anapore. The substrate may comprises a k*l substrate deepenings array of deepenings (with sample material), wherein k and l are independently in the range of 1-10,000, such as 2-10,2000, especially 2-5000. In a specific embodiment, the arrays of the substrate and the stamp can be aligned in a male-female configuration.

In an embodiment, the method of the invention further comprises aligning the substrate and the stamp before contacting the elevations with the sample material on the substrate (especially in an embodiment in one or more deepenings). Especially, the substrate or the stamp or both the substrate and the stamp may be rotatable in parallel planes, which may especially allow alignment.

In an embodiment, the elevations of the stamp have tops, wherein the tops form an elevations top plane, wherein the microscope allows, without changing the objective, a first lens configuration and a second lens configuration, wherein the first lens configuration allows a sharp image of at least part of the elevations top plane and wherein the second lens configuration allows a sharp image of at least part of the substrate at a distance between the substrate and the tops larger than 0 cm. More especially, in an embodiment the first or the second lens configuration are selectable by introducing a second lens between lens and an ocular. In a further specific embodiment, the elevations of the stamp have tops, wherein the tops form an elevations top plane, wherein the stamp has a second side, and wherein the elevations top plane and the second side are substantially parallel.

The method may in an embodiment further comprise transporting the stamp with samples to an analysis unit for analysis of one or more of the samples. The analysis unit may for instance be selected from the group consisting of a UV VIS spectrometer, luminescence spectrometer, fluorescence spectrometer and mass spectrometer. The method may alternatively or in addition also further comprise contacting the elevations with samples with one or more second substrates, especially contacting the elevations with samples with a plurality of second substrates (respectively). In this way, essentially the microscope of the invention may function as a micro-scale version of the velvet pad as used in classical microbiology to replicate patterns of micro organisms between agar surfaces.

In a specific embodiment, the plurality of elevations comprise elevations with different chemical or biological properties. In a variant, the plurality of elevations comprise elevations which differ from each other in respect of hydrophobicity or hydrophilicity. In a further specific embodiment, the plurality of elevations comprise elevations which are provided with one or more auxiliary compounds selected of the group consisting of acids, bases, antibodies, organic catalysts, inorganic catalysts, markers, receptor ligands, oligonucleotides, and DNA-binding proteins.

In order to seize the stamp, the optical microscope further comprises in an embodiment a vacuum pump and an adaptor arranged to receive the stamp and arranged to align at least part of the transparent part of the stamp in front of the objective, the adaptor further comprising a vacuum channel in connection with the vacuum pump, wherein the vacuum channel has an opening, and wherein arranging at least part of the transparent part of the stamp in front of the objective comprises attaching the stamp to the adaptor by means of vacuum force. Alternatively or additionally, the optical microscope further comprises an adaptor arranged to receive the stamp and arranged to align at least part of the transparent part of the stamp in front of the objective, the adaptor further comprising an (electro)magnet, and wherein arranging at least part of the transparent part of the stamp in front of the objective comprises attaching the stamp to the adaptor by means of magnetic forces. Alternatively or additionally, the adaptor may further comprise an electrostatic device. Such electrostatic device is arranged to seize the stamp and attach the stamp to the adaptor by electrostatic forces. Hence, in a specific embodiment, the invention also provides the adaptor further comprising one or more seizing devices, wherein the one or more seizing device are arranged to attach the stamp to the adaptor, and wherein the one or more seizing devices are selected from the group consisting of a vacuum channel, connectable to a vacuum pump, with opening, an (electro)magnet, and an electrostatic device. In an embodiment, the adaptor is arranged to allow attachment to the objective.

In a further aspect, the invention provides a method for taking a plurality of samples from a substrate with sample material by using an optical microscope and a stamp, wherein

the optical microscope comprises an objective with a lens, and a substrate holder, wherein a lens-holder distance between the lens and the substrate holder is variable, and

the stamp comprises at a side a plurality of elevations and wherein at least part of the stamp is transparent, wherein the method comprises:

arranging at least part of the transparent part of the stamp in front of the objective, with the plurality of elevations directed to the substrate holder;

arranging the substrate with sample material to the substrate holder

optionally aligning the stamp and the substrate, especially in a male-female configuration; and

contacting the elevations with the sample material, thereby providing the plurality of elevations with samples, and wherein aligning the stamp and the substrate and/or (the actual) contacting the elevations with the sample material is preferably controlled by inspection through the optical microscope; and

preferably subjecting the samples to a process selected from the group consisting of analysis and replication.

The invention further provides a kit comprising an adaptor designed to attach to an objective of an optical microscope, and the stamp, as described herein. As mentioned above, the adaptor may further comprise a vacuum channel, connectable to a vacuum pump, with opening, and/or the adaptor may further comprise an (electro)magnet.

The invention further provides an optical microscope comprising an objective with a lens, an adaptor, and a substrate holder for a substrate, wherein the adaptor is arranged to receive the stamp comprising at a side a plurality of elevations and, wherein preferably the stamp comprises an n*m stamp elevations array of elevations, wherein n and m are independently preferably in the range of 2-10,000, wherein neighbouring elevations are preferably arranged at a shortest elevation distance in the range of 1-1000 μm, wherein the elevation height of the elevations is preferably in the range of 0.1-1000 μm, wherein at least part of the stamp is transparent, wherein the adaptor is further arranged to align at least part of the transparent part of the stamp in front of the objective. The microscope may further comprise a device for moving the substrate in the plane.

Advantageously, the invention may provide a set of devices which can replicate the pattern distribution of micro colonies of micro organisms from one solid or porous surface (substrate) to another (second substrate). Essentially, the device may function as a micro-scale version of the velvet pad as used in classical microbiology to replicate patterns of micro organisms between agar surfaces. To obtain such result, it is especially desired to move some of the original pattern to one or more new area(s) whilst maintaining the spatial relationships. This will enable the production of replicates that can be sacrificed for analytic purposes such as a metabolite analysis or detection of specific nucleotide sequences while allowing recovery of a clone or strain with desired functionality from the replicate. To this end, the invention provides the stamp as described herein, and the substrate, which can be aligned in a male female configuration, due to the elevations of the stamp and in an embodiment also the (corresponding) deepenings of the substrate.

The extremely high precision stamp (“replicator pad”) on a highly miniaturized scale may allow individual printing areas of 1 μm to 1 mm. The entire stamp may for instance be an array of elevations (from 2 to 100 000 elevations). The stamps may pick up a representative fraction of the organisms in a micro-colony cultured on a surface of the substrate and may print (transfers) some or all of them to one or more other surfaces (of second substrates) where they can be assayed or re-cultured (for example recreating the original distribution or pattern if desired). The surfaces will often be planar but not necessarily so; preferably however, the surfaces will substantially be planar. Advantageously, the invention allows non-cross contamination between printing areas and transfer to multiplexed assay. Examples of multiplexed assays include situations where the cells adhering to each elevation of the stamp are taken to a separate analysis. This may be a molecular analysis (for example a series of polymerase chain reactions) or other form of investigation or processing (for example cultivating the micro organisms on each elevation separately).

The method of the invention may especially be used in for instance high throughput and content screening and especially mutant selections, deploying and storage of libraries strains and mutants, and presenting organisms grown under different conditions to a multiplexed assay (e.g. PCR), i.e. sampling from an area and taking each point to a separate assay.

### BRIEF DESCRIPTION OF THE DRAWINGS

Embodiments of the invention will now be described, by way of example only, with reference to the accompanying schematic drawing in which corresponding reference symbols indicate corresponding parts, and in which:

FIGS. 1a-1e schematically depict embodiments of the stamp and substrate;

FIGS. 2a-2b schematically depict an embodiment of the microscope of the invention;

FIGS. 3a-3b schematically depict embodiments of the adaptor of the invention; and

FIG. 4 schematically depict an embodiment of the kit according to an embodiment of the invention;

FIGS. 5a-5c schematically depict applications according to embodiments of the method of the invention;

FIGS. 6a-6b depict an enlargement of part of a stamp and an enlargement of a printed area, respectively, according to an experiment.

### DESCRIPTION OF PREFERRED EMBODIMENTS

As mentioned above, the invention provides a method for taking a plurality of samples, a stamp, a kit comprising an adaptor and a stamp, and an optical microscope. In order to explain the aspects and embodiments of the invention, below first the stamp and substrate are described, respectively. Then the microscope and adapter, the kit, and the method of the invention are described, respectively.

### Stamp

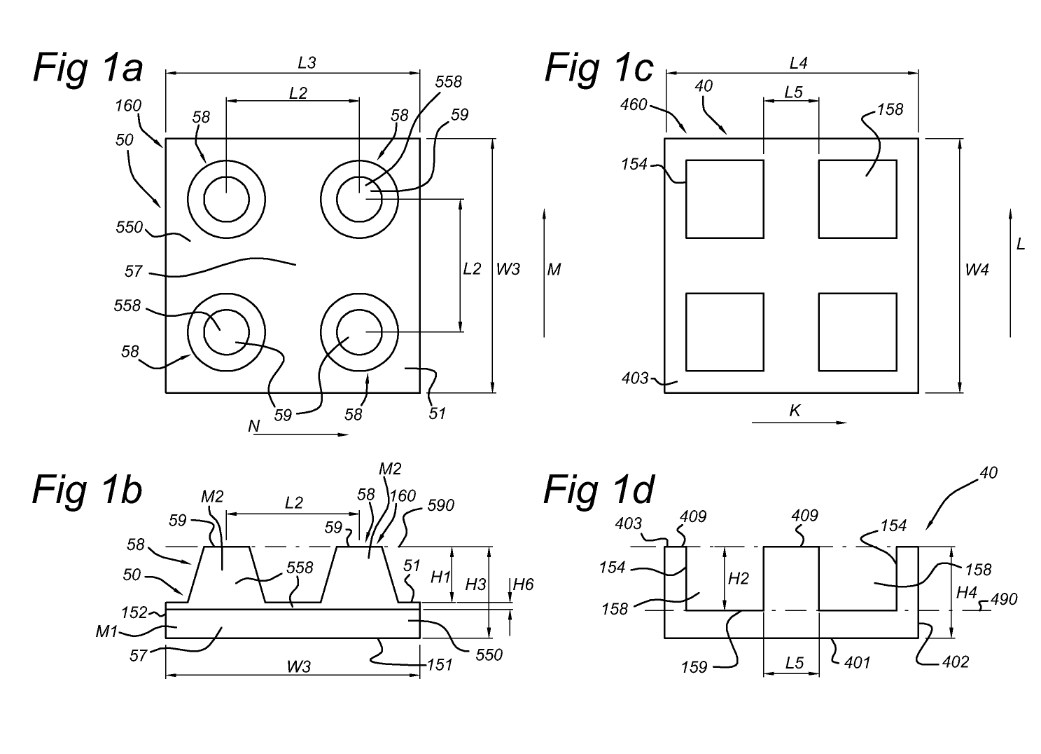

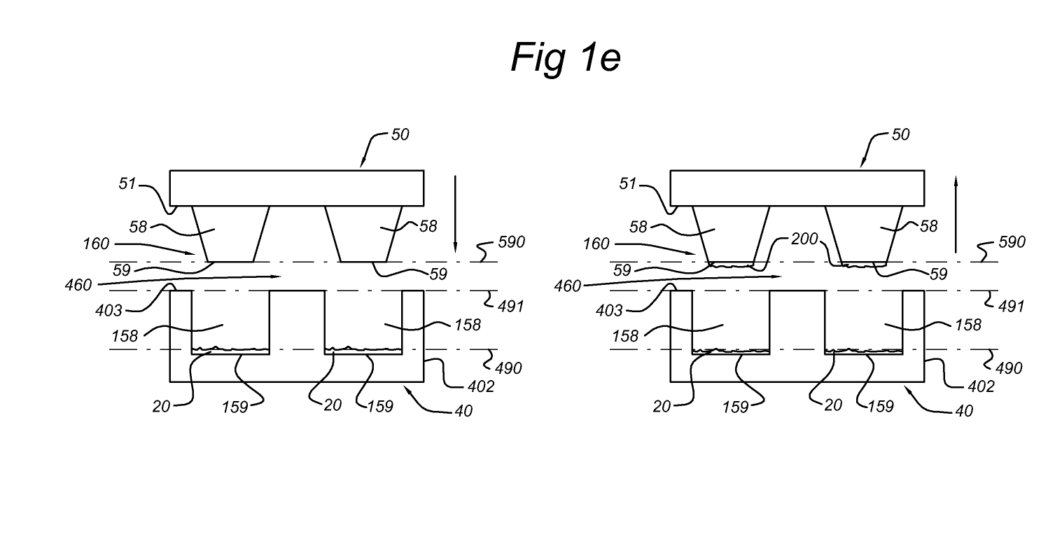

FIG. 1a schematically depicts in top view an embodiment of stamp 50. FIG. 1b schematically depicts in side view an embodiment of the stamp 50. The stamp 50, in this embodiment a square stamp, has a width W3, a length L3, and a height H3. Width W3, length L3, and height H3 are typically in the order of about 2-100 mm (such as 10 mm), about 2-100 mm (such as 10 mm), and about 0.2-20 mm (such as 0.6 mm), respectively. The stamp has a side 51, also indicated herein as first side, which can also be seen as “top side”. The stamp further has second side 151, which can also be seen as back side. In general, these sides 51 and 151 will be parallel. Further, the stamp 50 has edges 152. The side 51 further comprises elevations 58. These are in this embodiment arranged as an n×m array 160. Typically, the array of elevations comprises in the range of n=1-10,000, especially 2-10,000, more especially 2-5,000 and of m=10-10,000, especially 2-10,000, more especially 2-5,000. Hence, the stamp 50 comprises at the side 51 a plurality of elevations 58.

Each elevation 58 has a top 59. Especially, the tops 59 of the elevations 58 are substantially flat surfaces, which are in general essentially parallel to the first side 51 and/or which are in general essentially parallel to the second side 151. In this way, the tops 59 of the elevations 58 are substantially all in a plane 590, also indicated as elevations top plane. In a specific embodiment, the elevations top plane 590 and the second side 151 are substantially parallel.

The elevations 58 will in general have dimensions in the order of 1-1000 μm*1-1000 μm at the bottom and in the order of 1-1000μm*1-1000 μm at the tops 59; i.e. the length and the width or the diameter are at the bottom and at the top in the order of 1-1000 μm. The elevations may be straight or may be (partly) tapered (as schematically shown in FIG. 1b). In general, the length and the width, or the diameter of the elevations 58 at the bottom and at the top smaller than the length and width or diameter, respectively, of the corresponding cavities of the substrate (see below) to be used. The height of the elevations 58, indicated in FIG. 1b as elevation height H1, is in an embodiment the order of 1-1000 μm. The shortest distance between two tops 59, indicated in FIGS. 1a and 1b as L2, will in general be in the range of about 2-500 μm. In case the tops 59 are flat, the distance between centres of adjacent tops 59 are taken as shortest elevation distance.

At least part of the stamp 50 is transparent. This part is indicated as part 57. The stamp 50 with transparent part 57 may be for instance a stamp with a hole (through which in an embodiment visible inspection is possible, such as visible inspection through a microscope), especially a plurality of holes (at least 2), but may in another embodiment be a stamp which comprises a material that is transparent. In a specific embodiment, the entire stamp 50 is made of a transparent material. In an embodiment, the stamp 50 substantially consists of a transparent material selected from the group consisting of PMA (polymethacrylate), PMMA (polymethylmethacrylate), PC (polycarbonate), PDMS (polydimethyl siloxane), perspex, quartz, gemstones, glass, ceramic materials (such as transparent ceramics). Transparent herein means that the stamp 50 comprises at least part 57, wherein visible inspection through the stamp in the direction from side 51 to second side 151 is possible. Such stamps 50 may for instance be obtained by using a corresponding mould and curing PMA, PMMA or other transparent polymers in such mould. Alternatively, such stamps 50 may be obtained by hot embossing such transparent materials.

Stamps may also comprise a plurality of materials, such as a base part, for instance essentially consisting of glass, and a top part comprising elevations, for instance essentially consisting of PDMS. Hence, in an embodiment, the stamp comprises two or more materials. In a specific embodiment, the stamp comprises a base part 550, comprising a first material m1, especially a transparent material, such as glass or perspex, especially glass, and an elevation part 558, comprising the elevations 58, and comprising a second material m2, such as PDMS. Especially, the base part 550 consists (at least partly) of a transparent material. Further, especially also the elevation part 558 consists of a transparent material. Such stamps 50 may for instance be made by curing a polymeric material (as indicated above, such as PDMS) on a Si wafer with a counter configuration, arranged to generate an elevation part 558, and attached the thus created elevation part 558 to the base part 550. FIG. 1b schematically depicts an embodiment, with the elevation part 558 comprising the elevations 58, and base part 550. As a kind of film comprising the elevations 58, the elevation part 558 may be attached to the base part 550. The elevation part 558 may have a base height H6 of about 20 μm-5 mm.

### Substrate

To take a plurality of samples, the stamp 50 according to the invention may advantageously be used. Substantially any substrate can be used, but especially substrates designed to provide a male-female construction with the stamp 50 may advantageously be applied. Such array type substrates may especially be used in high throughput (HTP) screening. Hence, the substrate may be a patterned substrate, such as suitable for a male-female arrangement with the stamp, but the substrate may also be substantially flat, including porous substrates.

FIGS. 1c and 1d schematically show an embodiment of the substrate suitable for use in the method of the invention, in top view and side view, respectively. The substrate is indicated with reference number 40. The substrate 40 may in specific embodiments be a substrate as described in the embodiments of WO2006112709 and WO2006112713, which are herein incorporated by reference.

The substrate 40, in the schematically depicted embodiments a square substrate, has a width w4, a length L4, and a height H4. Width w4, length L4, and height H4 are typically in the order of 2-200 mm, 2-200 mm, and 1-50 mm, respectively. The substrate 50 has a side 403, also indicated herein as first substrate side, which can also be seen as “top side”. The substrate 40 further has second substrate side 401, which can also be seen as back side. In general, these sides 403 and 401 will be parallel. Further, the substrate 40 has edges 402. The side 402 may further preferably comprise deepenings 158. The term “deepenings” especially refers to wells or compartments. These are in this embodiment arranged as an k×1 array 460. Typically, the array 460 of deepenings 158 comprises in the range of n=2-10,000 and of m=2-10,000 deepenings 158. Hence, the substrate 40 comprises at the side 403 a plurality of deepenings 158.

Each deepening 158 has a bottom 159. Hence, in an embodiment, substrate 40 has deepenings 158 with bottoms 159, and thereby also forms side 403 with tops 409. The tops 409 are the elevations between the deepenings 158 and form a plane 491. In general, the tops 409 form plane 491 and side 403 essentially consist of the tops 409. Especially, the bottoms 159 of the deepenings 158 are substantially flat surfaces, which are in general essentially parallel to the top side 403 and/or which are in general essentially parallel to the bottom side 401. In this way, the bottoms 159 of the deepenings 158 are substantially all in a plane 490, also indicated as deepening bottom plane. In a specific embodiment, the deepening bottom plane 490 and the side 403 are substantially parallel.

The deepenings 158 will in general have dimensions in the order of 1-1000 μm*1-1000 μm; i.e. the length and the width or the diameter of the deepenings 158 are at the bottom and at the top in the order of 1-1000 μm; especially in the range of 2-1000 μm. The height of the deepenings 158, indicated in FIG. 1c as height H2, is in an embodiment the order of 0.1-1000 μm. The shortest distance between two deepenings 158, indicated in FIGS. 1c and 1d as L5, will in general be in the range of about 1-1000 μm. The deepenings may be straight (as schematically shown in FIG. 1d) or may be partly tapered. In general, the length and the width, or the diameter of the deepenings 158 at the bottom and at the top are larger than the length and width or diameter, respectively, of the corresponding elevations 58 of the stamp 50 (see above) to be used.

Especially, the substrate 40 comprises a porous substrate, and even more especially, the substrate is a porous substrate, such as an anopore substrate. An advantage of using a porous substrate is that sample material such as bacteria, virus, fungi, yeast and any other living cell or tissue, may be fed through the pores of the substrate (from beneath the substrate).

In a specific embodiment, the substrate 40 comprises a biochip comprising a porous support, wherein the porous support comprises at least one surface coated with a coating, wherein the coating is patterned with a micro compartments pattern, with the support providing a bottom surface to the compartments and the coating providing edges to the compartments, and wherein the pattern comprises at least 400 compartments per mm². Especially, the bottom 159 surface area of the compartments is in the range of 20 μm²-20,000 μm² and wherein the edges, indicated with reference number 154, of the compartments have a height in the range of 0.2-1000 μm. In a specific embodiment, the pattern comprises at least 10,000 compartments per mm², even more especially, at least 100,000 compartments per mm². Especially, the pattern comprises 100-200,000 compartments per mm², even more especially, 1000-200,000 compartments per mm².

FIG. 1e schematically depicts a detail of part of the method of the invention (see also below). The left figure schematically depicts the stamp 50 and substrate 40 before the stamp has had contact with sample material, indicated with reference number 20. The stamp 50 can be moved to the substrate 40, or vice versa, or both can be moved to each other, and the stamp 50 contacts the sample material 20, i.e. at least part of the tops 59 of at least part of the total number of the elevations 58 is brought into contact with at least part of the sample material in at least part of the total number of deepenings 158. By contacting, at least part of the sample material 20 will stick to at least part of the tops 59 of at least part of the total number of elevations 58 of stamp 40. This sampled sample material is indicated with reference number 200 (see right figure) and is further indicated as sample(s) 200. Then, the stamp 50 is moved away from substrate 40, or vice versa, or both are moved away from each other.

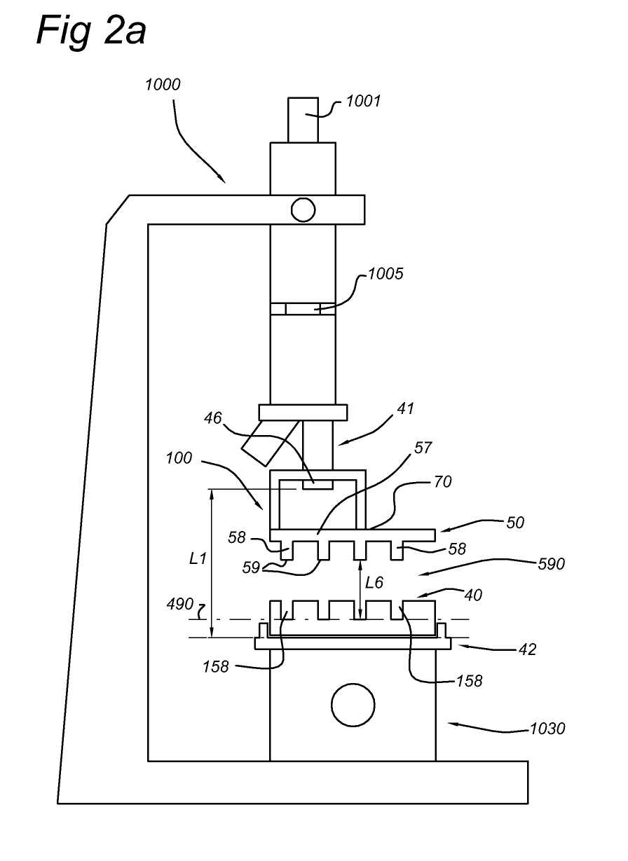

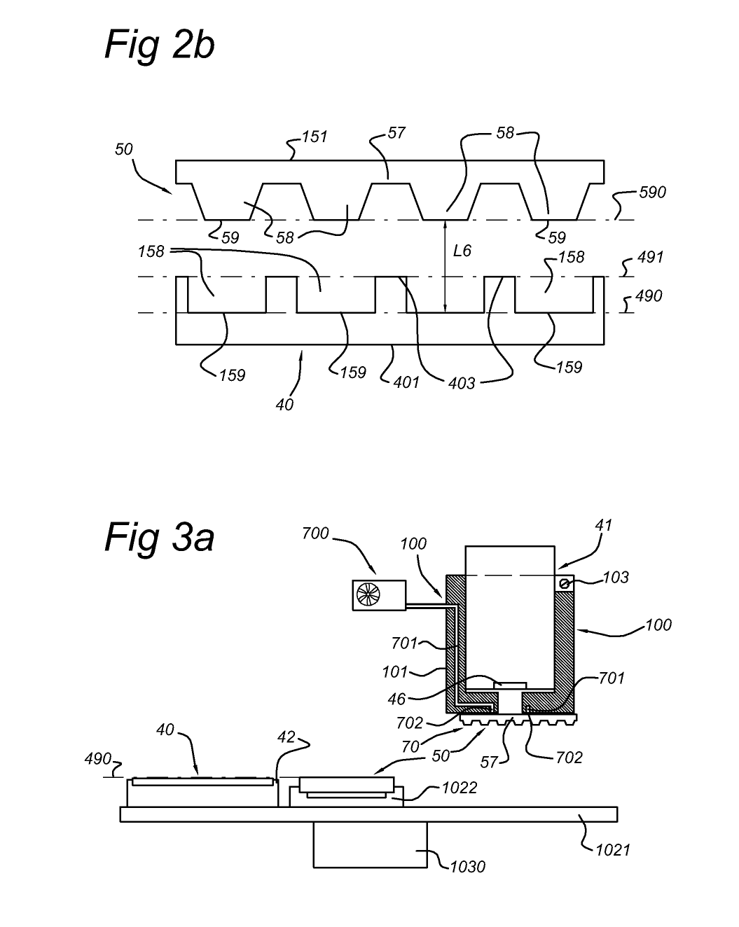

As shown in the drawings in 1e, especially both stamp 50 and substrate 40 are constructed to be arranged with substantially parallel planes 590 and 490, respectively. In yet another embodiment, especially both stamp 50 and substrate 40 are constructed to be arranged with substantially parallel planes 590 and 490, respectively. Yet in a further embodiment, the arrays 460 and 160 of the substrate 40 and the stamp 50 are alignable (i.e. can be aligned) in a male-female configuration. Hence, the deepenings 158 of substrate 40 are constructed to receive at least part of the elevations 58 of the stamp 50, and the elevations 58 of stamp 50 are constructed to fit in at least part of the deepenings 158 of substrate 40. A detail figure is shown in FIG. 2b.

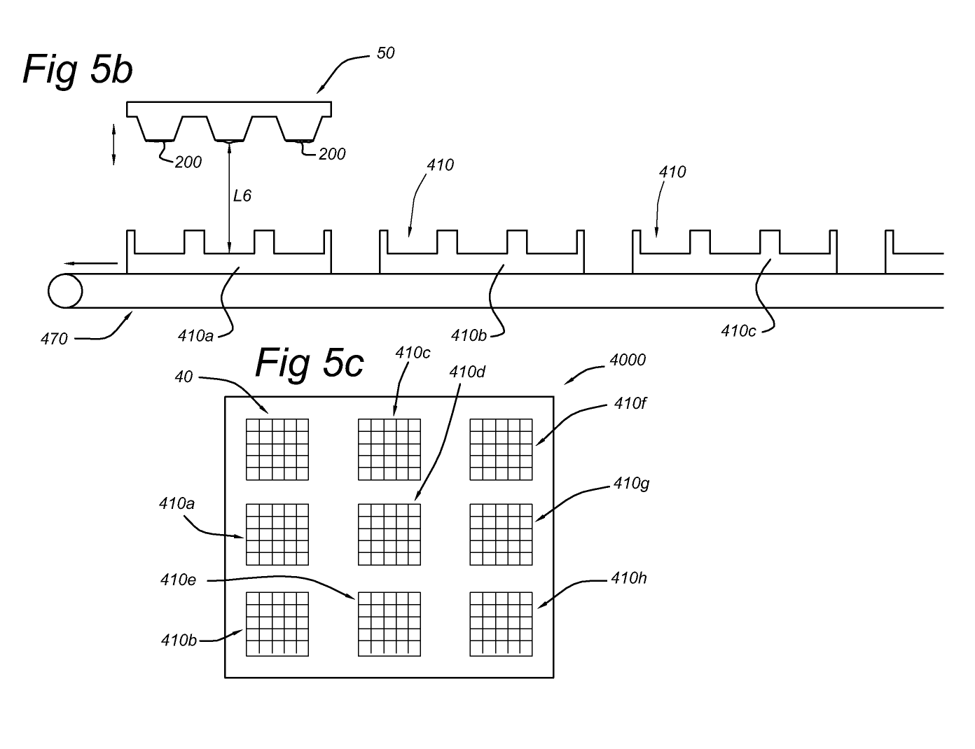

Another embodiment of the substrate is schematically depicted in FIG. 5c, see below.

### Microscope & Adaptor

Having described some embodiments of the stamp 50 of the invention and the substrate 40, now embodiments of the microscope, indicated with reference number 1000, of the invention are described. Herein, the term “microscope” especially refers to an optical microscope. Hence, the optical microscope 1000 is a microscope for visible light.

FIG. 2a schematically depicts an embodiment of the microscope 1000 of the invention. In general, only elementary parts of the microscope 1000 are schematically depicted. The microscope 1000 is an optical microscope which comprises an objective 41 with a lens 46. Herein, the term lens 46 may include a plurality of lenses, respectively. Further, the microscope 1000 comprises an adaptor 100, which may in an embodiment be attached to the objective 41, and a substrate holder 42 for a substrate 40. Embodiments of the substrate 40 have been described above, and the substrate holder 42 may be any substrate holder. Especially, the substrate holder 42 is arranged to be movable in one or more directions. The substrate holder may be arranged to a transportation device 1030, such as a substrate table, which allows moving the substrate 40 in one or more directions, especially in the direction of the objective 41, but in other embodiments (also) in a plane parallel to the plane of deepening bottoms 490 (when the substrate 40 is arranged to the substrate holder 42). Hence, the microscope 1000 especially comprises an embodiment of the transportation device 1030 for moving the substrate 40 in the plane 490.

Especially, the substrate 40 is movable (while being arranged to the substrate holder 42), in all directions. In an embodiment, the transportation device 1030 includes an xyz-table, i.e. the transportation device 1030 is constructed to be able to move the substrate holder 42 in x-, y- and z-directions; optionally also including (partial) rotation in the xy-plane).

The adaptor 100 is a device that is arranged to position the stamp 50 in front of the objective 41, more precisely in front of the lens 46 of the objective 41. Therefore, the adaptor 100 is especially arranged to align at least part of the transparent part 57 of the stamp 50 in front of the objective 41. To this end, the adaptor 100 may further comprise one or more seizing devices 70 to attach the stamp 50 to the adaptor 100 (see below). The adaptor 100 is arranged to hold the stamp 50 (comprising at side 51 a plurality of elevations 58 and wherein at least part 57 of the stamp 50 is transparent). In this way, the adaptor 100 may position the stamp 50 in front of the objective, in such a way that the transparent part 57 is in front of the lens 46.

In FIG. 2a, some further aspects of the microscope are shown. For instance, an ocular 1001 is depicted, which can be used for visible inspection. Alternatively, ocular 1001 may be replaced by a CCD or CMOS camera, or another digital camera. Such options are known in the art.

The distance between the lens 46, herein also indicated as last lens, and the substrate holder 42 is indicated with reference L1. This lens-holder distance L1 is variable. This distance may be variable due to an embodiment of the microscope 1000 wherein the transportation device 1030 is arranged to be able to move the substrate 40 in the direction of the objective 41, or this lens-holder distance L1 may be variable due to an embodiment wherein the microscope 1000 is arranged to be able to move the objective 41 (and optionally other parts) in the direction of the substrate table 42, or wherein the microscope 1000 is arranged to allow both options to decrease the lens-holder distance L1.

When stamp 50 is attached to the adaptor 100, visible inspection through transparent part 57 by eye or CCD or CMOS camera (or other type of digital camera) is possible. Assuming the lens-holder distance L1>0 cm, when reducing the lens-holder distance L1, the distance between stamp 50 and substrate 40 decreases. Such decrease may be monitored by inspection through the microscope 1000. At a certain point, the elevation 59 of stamp 50 touch the sample material 20 and/or the bottom 159 of the deepenings 158 of the substrate. This whole process can be monitored through the microscope. Hence, the invention provides a method wherein contacting the elevations 58 with the sample material 20 is controlled by inspection through the optical microscope 1000. As mentioned above, such inspection might be “directly” by eye, or by using a CCD or CMOS camera, or another type of digital camera. Note that the latter option may allow a full automization of the process of contacting.

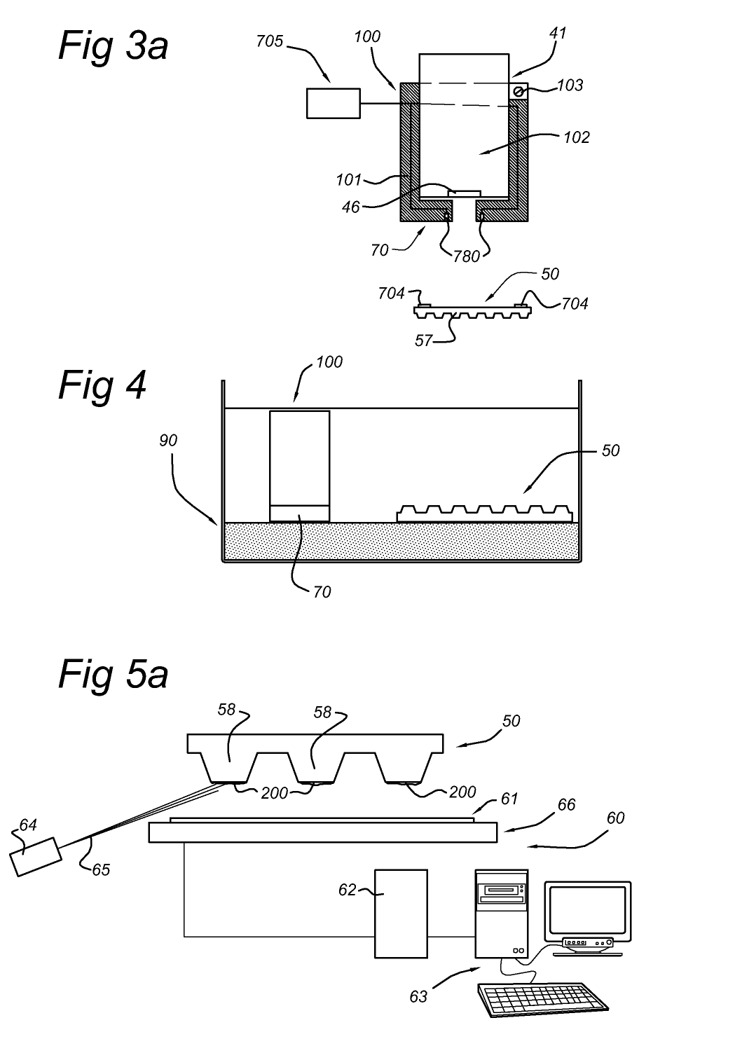

In order to allow the microscope delivery of sample material 20 to the elevations 59 of the stamp, the microscope 1000 has in embodiment to be equipped with a device to arrange the stamp 50 in front of the objective 42. Herein, the term “in front of” may include embodiments wherein there is some space between the last lens 46 and the stamp 50 (especially side 151 of stamp 50), but may also include embodiments, wherein the stamp 50 is in touch with the lens 46. To this end, the adaptor 100 is provided (see also above). In an embodiment, the adaptor 100 is arranged to be attachable to the objective 41. In a further specific embodiment of the microscope 1000 according to the invention, the adaptor 100 is attached to the objective 41. Some embodiments of the adaptor 100 are schematically depicted in FIGS. 3a and 3b. Here, the embodiments include adaptors 100 which are attached to objective 41.

The distance between the stamp 50, more precisely the tops 59 of elevations 58 to the substrate is indicated as L6. As mentioned herein, the substrate may comprise deepenings 158. Hence, in such embodiments, the distance L6 between the stamp 50 and the substrate 40 is especially defined as the distance between the tops 59 of the elevations 58 and the bottoms 159 of the deepenings 158, especially in an aligned configuration (i.e. male-female configuration, as schematically depicted in FIG. 2a). When the substrate 40 is substantially flat, the distance L6 is the distance between the tops 59 of the elevations 58 and the substrate 40.

By decreasing the distance L6, one or more elevations 58, more precisely one or more elevations tops 59, will contact the sample material 20, thereby providing one or more samples 200 to one or more elevations 58.

The phrase “wherein contacting the elevations 58 with the sample material 20 is controlled by inspection through the optical microscope 1000” especially indicates that aligning the stamp 50 and substrate 40, especially in a male-female configuration, and/or contacting the elevations with the sample material 20, is controlled by inspection through the microscope 1000.

Hence, in an embodiment, the phrase “contacting the elevations 58 with the sample material 20, thereby providing the plurality of elevations 58 with samples 200, wherein contacting the elevations 58 with the sample material 20 is controlled by inspection through the optical microscope 1000” relates to an embodiment comprising “aligning the stamp 50 and the substrate 40, contacting the elevations 58 with the sample material 20, thereby providing the plurality of elevations 58 with samples 200, wherein aligning the stamp 50 and the substrate 40, especially in a male-female configuration, is controlled by inspection through the optical microscope 1000”.

Hence, in another embodiment, the phrase “contacting the elevations 58 with the sample material 20, thereby providing the plurality of elevations 58 with samples 200, wherein contacting the elevations 58 with the sample material 20 is controlled by inspection through the optical microscope 1000” relates to an embodiment comprising “optionally aligning the stamp 50 and the substrate 40, and contacting the elevations 58 with the sample material 20, thereby providing the plurality of elevations 58 with samples 200, wherein (the actual) contacting the elevations 58 with the sample material 20 is controlled by inspection through the optical microscope 1000”.

Hence, in yet another embodiment, the phrase “contacting the elevations 58 with the sample material 20, thereby providing the plurality of elevations 58 with samples 200, wherein contacting the elevations 58 with the sample material 20 is controlled by inspection through the optical microscope 1000” relates to an embodiment comprising “aligning the stamp 50 and the substrate 40, contacting the elevations 58 with the sample material 20, thereby providing the plurality of elevations 58 with samples 200, wherein aligning the stamp 50 and the substrate 40, especially in a male-female configuration, and (the actual) contacting the elevations 58 with the sample material 20 is controlled by inspection through the optical microscope 1000”.

FIGS. 3a-3b schematically depict part of the objective 41 and embodiments of the adaptor 100. The adaptors in FIGS. 3a and 3b comprise a body 101 with a (tubular) cavity 102. The adaptor 100 in these embodiments is designed to be slided over at least part of the objective 41, thereby circumferentially surrounding the objective 41. A setscrew 103, or other means known in the art, may be used to attach the adaptor 100 to the objective 41 or keep the adaptor 100 attached to the objective 41. To receive the stamp 50, the adaptor 100 may further comprise one or more seizing devices 70. Such seizing device(s) 70 is (are) arranged to hold the stamp 50 at the desired position attached to the adaptor 100. Especially, the seizing devices allow a predetermined temporarily holding. For instance, in specific embodiments the seizing device 70 is selected from the group consisting of a vacuum channel 701 with opening 702, the vacuum channel connectable to a vacuum pump 700, and an (electro)magnet 780.

FIG. 3a schematically depicts an embodiment wherein the optical microscope 1000 (only parts of the microscope 1000, such as objective 41, are shown) further comprises vacuum pump 700 and the adaptor 100, which, as indicated above, is arranged to receive the stamp 50 and arranged to align at least part of the transparent part 57 of the stamp 50 in front of the objective 41b. The adaptor 100 further comprises vacuum channel 701 in connection with the vacuum pump 700, wherein the vacuum channel 701 has an opening 702. The vacuum channel 701 can be integrated in the body. Further, the term “opening” may include a plurality of openings. At least of the transparent part 57 of the stamp 50 can be arranged in front of the objective 41 by attaching the stamp 50 to the adaptor 100 by means of vacuum force. For instance, the stamp 50 and the adaptor 100 are arranged in a configuration that the adaptor 100, especially the seizing device 70, is over the stamp 50, especially over side 151 of stamp 50. Optionally, in an embodiment inspection through the microscope 1000 may be used to align the adaptor 100 over stamp 50, especially over side 151. Then, the vacuum force may be increased and/or the distance between adaptor and stamp 50 may be reduced in order to attach the stamp 50 to adaptor 100. By maintaining a vacuum force above a threshold such that the stamp 50 is hold (maintains attached), the stamp 50 is aligned in front of objective 41.

In a further embodiment, the adaptor 100 may further comprise an (electro)magnet 780. Here, arranging at least part of the transparent part 57 of the stamp 50 in front of the objective 41 comprises attaching the stamp 50 to the adaptor 100 by means of magnetic forces. FIG. 3b schematically depicts an embodiment wherein the one or more (electro)magnets 780 are used as seizing device 70 (attached to adaptor 100). As will be clear to the person skilled in the art, the stamp 50 will have one or more magnets arranged to allow a magnetic holding by the (electro)magnet(s) 780 of the seizing device 70 of the stamp 50 (with in this embodiment counter magnets or magneto materials). The counter magnets or magneto materials (Fe or Ni containing metallic compounds or metal oxides that can be attracted by magnets) are indicated in the FIG. 3b with reference 704. The microscope 1000 may hereto further comprise a voltage source 705, arranged to apply a voltage to the (electro)magnet(s) 780 to generate magnetic forces for seizing the stamp 50 (with counter magnets or magneto materials 704). By maintaining the magnetic force above a threshold such that the stamp 50 is hold (maintains attached), the stamp 50 is aligned in front of objective 51. In a specific embodiment, the one or more magnets 780 are electromagnets, arranged to seize the stamp 50, which stamp comprises magneto materials 704 arranged to be attracted by the magnetic forces of the electromagnets 780.

FIGS. 3a and 3b further depict a specific embodiment, wherein a microscope table 1021 is movable by means of transportation device 1030. Different equivalent configurations are possible, but the embodiment schematically depicted in FIG. 3a comprises microscope table 1021, which is movable by transportation device 1030, and which comprise a stamp holder 1022 and substrate table 42. Since in this embodiment the microscope table 1021 is movable, this configuration allows an arrangement wherein the stamp holder 1022 is arranged under the adaptor 100 (i.e. under objective 41) and an arrangement wherein the substrate holder 42 is arranged under the adaptor 100 (i.e. under objective 41). In this way, a method can be applied comprising arranging the adaptor 100 in proximity of stamp 50, seizing the stamp 50 by the seizing device 70, thereby arranging the stamp 50 in front of the objective 41, arranging the substrate 40 under the stamp 50 and contacting the stamp 50 with the sample material 20 on the substrate. Note that alternatively or additionally, also the microscope 1000 and/or stamp 50 may be movable in the xy-plane relative to the substrate 40.

For a good alignment, in an embodiment the stamp 50, i.e. especially the stamp 50 with an array 160 of elevations 58, and the substrate 40, i.e. especially the substrate with an array 460 of deepenings 158, are alignable in a male-female configuration, especially with the stamp 50 and/or the substrate 40 rotatable in parallel planes. Aligning one elevation 58 to one deepening 158, by visual inspection through the microscope might be relatively easy, but in order to have all the elevations 58 penetrate in the deepenings 158, a further alignment may be necessary. Hence, in an embodiment the method further comprises aligning the substrate 40 and the stamp 50 before contacting the elevations 58 with the sample material 20. This may be done by applying transportation device 1030, especially a transportation device that allows movement of the substrate table in a plane parallel to the plane 490 of the deepenings 158. Hence, as mentioned above, in an embodiment the invention further comprises the device 1030 for moving the substrate 40 in the plane 490. In a specific embodiment, the substrate 40 or the stamp 50 or both the substrate 40 and the stamp 50 are rotatable in parallel planes 490 and 590, respectively. For instance, adaptor 100 may be rotatable, for instance rotatably attached to objective 41.

### Kit

The adaptor 100 according to the invention may advantageously be applied to standard optical microscopes. Hence, the method of the invention may especially be applied with standard microscopes in combination with the adaptor 100 according to the invention and stamp 50 according to the invention. To that end, the invention also provides, as schematically shown in FIG. 4, a kit 90 comprising an adaptor 100 designed to attach to an objective 41 of an optical microscope 1000 and a stamp 50 according to the invention. As mentioned above, in an embodiment, the adaptor 100 further comprises vacuum channel 701, connectable to a vacuum pump 700, with opening 702, and in another embodiment, the adaptor 100 further comprises (electro)magnet 780.

### Method for Taking a Plurality of Samples

Having discussed the stamp 50, substrate 40, adaptor 100 and microscope 1000, now in more detail the method for taking a plurality of samples of the invention is described. The method of the invention may especially be applied with the optical microscope 1000 and/or the stamp 50 and/or the substrate(s) 40 according to the invention.

The method of the invention involves taking a plurality of samples 200 from the substrate 40 (be it an array of deepenings or not) with sample material 20 by using the optical microscope 1000 and the stamp 50. At east part of the transparent part 57 of the stamp 50 is arranged in front of the objective 41, with the plurality of elevations 58 directed to the substrate holder 42, and the substrate 40 with sample material 20 is arranged to the substrate holder. Optionally, the substrate 40 and the stamp are aligned 50 before contacting the elevations 58 with the sample material 20. The phrase “the substrate 40 and the stamp 50 are aligned” and similar phrases refer to embodiments wherein the substrate 40 is aligned to the stamp 50, or the stamp 50 is aligned to the substrate or both are aligned to each other. After the optional alignment, the elevations 58 are contacted with the sample material 20, thereby providing the plurality of elevations 58 with samples 200.

The sample material 20 may be any sample material 20, but the method of the invention may especially be applied with sample material selected from the group consisting of bacteria, virus, fungi, yeast and any other living cell or tissue. Note that in general the samples 200 on the elevations 58 will be the same sample material 20 as on the substrate 40, i.e. at least part of the sample material 20 present in a deepening 158 will be sampled to the corresponding elevation 58 as sample 200. Note that different deepenings 158 may comprise different types of sample material 20. However, when the elevations 58 have specific (chemical or biological) properties, the sample 200 may at least partially differ from the corresponding sample material 20 (for instance due to a chemical reaction as). Herein the term “corresponding” refers to a specific deepening 158 receiving a specific elevation 58.

Hence, in an embodiment, the plurality of elevations 58 comprise elevations 58 with different chemical or biological properties. In a specific embodiment, the plurality of elevations 58 comprises elevations 58 which differ from each other in respect of hydrophobicity or hydrophilicity. In yet another specific embodiment, the plurality of elevations 58 comprise elevations 58 which are provided with one or more auxiliary compounds selected from the group consisting of acids, bases, antibodies, organic catalysts, inorganic catalysts, markers, receptor ligands, oligonucleotides, and DNA-binding proteins. As will be clear to the person skilled in the art, especially the tops 59 of the elevations may have specific chemical or biological properties, in the sense that the tops 59 may differ in hydrophobicity or hydrophilicity and/or are provided with the auxiliary compound(s).

Having brought the tops 59 of the elevations into contact with the sample material 20 (in the deepenings 158), stamp 50 with sample material(s) 200 on at least part of the total number of elevations 58 is obtained. In this way, a plurality of samples 200 are obtained. These sample(s) may be subjected to a number of processes, like analysis, treatment and replication. Before and/or after analysis or replication, at least part of the total number of samples 200 may be treated. Replication and analysis are below shortly discussed. As will be clear to the person skilled in the art, replication may be preceded by analysis but after replication, the replicated samples may also be subjected to an analysis. An advantage of the method of the invention is that the method allows a relative high throughput of a plurality of samples 200. Treatment may for instance include lysis, induction of molecules within the target cells, alterations in nutritional state, subjecting cells to stresses or protective treatments.

Note that sample material 20 may be obtained from one part of the substrate 40 and may be replicated (as sample(s) 200) on another part of the substrate 40. Hence, the invention provides in an embodiment also a method comprising contacting the elevations 58 with samples 200 with another part of the substrate 40 than from the part where the sample material 20 was retrieved from; also in this way samples 200 may be replicated. As mentioned above, the substrate 40 may be patterned with deepenings 158 but may also be substantially flat. When the substrate 40 comprises deepenings 158, the deepenings 158 are preferably arranged to allow a male-female configuration with the stamp 50. However, this does not exclude that the number of deepenings 158 on the substrate 40 is larger than the number of elevations 58 on the stamp 50. In this way, providing the elevations 58 with sample material 200 and replicating the sample material 200 may be performed on one and the same substrate 40.

In a specific embodiment, the method of the invention further comprises an analysis, especially in the form of transporting the stamp 50 with samples 200 to an analysis unit 60 for analysis of one or more of the samples 200. An example is schematically shown in FIG. 5a. After having contacted the elevations 58 with sample material 20, the stamp 50 comprises elevations 58 with sample material 200. The distance between the stamp 50 and substrate 40 is increased, and a detector might be placed in front of the stamp 50. Optionally, the stamp 50 is removed from the adaptor and/or the adaptor 100 and stamp 50 are removed from the microscope 1000 and the 50 is placed in front of a detector.

FIG. 5a schematically shows an embodiment of the detector, here, the detector is indicated with reference number 66, and comprises in this embodiment a CCD array or CMOS camera 61. The sample(s) 200 are illuminated by a light source 64 which generates a beam of light 65. The detector 66 is part of an analysis unit 60, which may for instance be selected from the group consisting of a UV VIS spectrometer, luminescence spectrometer, fluorescence spectrometer and mass spectrometer. FIG. 5a shows a spectrometer which allows excitation of excitable material by light source 64 and detection of emission by detector 66. The analysis unit 60 comprises in this embodiment a detector 66, an optional detector signal converter 62 such as a spectrometer, which converts the signal of the detector 66 into data such as spectra, like excitation and emission spectra, and a computer 63, which may display the data from the detector 66 or optional detector signal converter 62. In this way, high throughput analysis may be performed. In an embodiment, the detector 66 may also comprise a fibre for receiving light which is arranged to scan at least part of the total number of plurality of elevations 58.

Whether or not the sample(s) 200 have been analysed, as for instance described above, the method of the invention may (also) further comprise contacting the elevations 58 with samples 200 with one or more second substrates 410. An example of such embodiment is schematically depicted in FIG. 5b. FIG. 5b schematically depicts stamp 50 and transporter 470. Transporter 470 is arranged to transport one or more second substrates 410 in such a way that stamp 50 can be used to replicate samples 200 on one or more second substrates 410. For instance, transporter 410 arranged second substrate 410a below stamp 50, the stamp 50 and substrate 410 may be aligned, and stamp 50 may “print” sample material 200 on substrate 410a. Then, the transporter moves, and a next second substrate 410b is arranged below stamp 50. Again, stamp 50 and next second substrate 410b may be aligned, and stamp 50 may “print” sample material 200 on substrate 410b, etc. Note that also equivalent transporters and replication methods may be used. Instead of the type of conveyer belt as schematically depicted, also robot arms or other type of transporting means may be used. Further, instead of a transporter that transports the second substrates 410, also a moving stamp 50 may be used, i.e. a transporter 470 is used that is arranged to arrange the stamp over a plurality of second substrates 410, sequentially. Combinations of transporters 470 may also be used, wherein both the stamp 50 and the second substrates 410 are moved, i.e. brought in the vicinity of each other, such that replication may be performed. Further, instead of, or in addition to a method wherein the distance L6 between stamp 50 and second substrate 410 (and substrate 40) is decreased by moving the stamp 50 to the second substrate 410 (and substrate 40), also an embodiment of the transporter 470 (or transportation device 1030) may be applied wherein the second substrate 410 (and substrate 40) move in the direction of the stamp 50. As mentioned above, replication might also take place at the same substrate 40 (but at another part) where form the sample material 200 has been retrieved.

After replication, the replicated material (or reaction products) thereof, may be analysed, optionally also with the microscope 1000.

In FIG. 5b the distance between the stamp 50, more precisely the tops 59 of elevations 58 to the second substrate 410 is also indicated as L6. As mentioned herein, the second substrate 410 may especially comprise deepenings 158. Hence, in such embodiments, the distance L6 between the stamp 50 and the second substrate 410 is especially defined as the distance between the tops 59 of the elevations 58 and the bottoms 159 of the deepenings 158, especially in an aligned configuration (i.e. male-female configuration, as schematically depicted in FIG. 5b).

In a further embodiment, the microscope 1000 is arranged to allow, without changing the objective 41, a first lens configuration and a second lens configuration, wherein the first lens configuration allows a sharp image of at least part of the elevations top plane 590 and wherein the second lens configuration allows a sharp image of at least part of the substrate 40 at a distance between the substrate 40 and the tops 59 larger than 0 cm. In a specific embodiment, the first or the second lens configuration are selectable by introducing a second lens 1010 between lens 46 and an ocular 1001. The microscope 1000 may for instance be equipped with an opening 1005, which may for instance also be used for filters. This opening may also be used to introduce the second lens 1010. An advantage of this embodiment is that contacting the elevations 58 with the substrate 40, may even be better controlled. For instance, when the lenses in the microscope 1000 and the stamp 50 are arranged to have a focal distance of the microscope within the elevations top plane 590, when bringing the elevations 58, i.e. the tops 59 of the elevations, and substrate 40 (especially the sample material 20 on the substrate 40) in contact with each other, only when the substrate 40 and tops 59 are very close, the substrate 40 will be observed. This might be less desired, in view of damage to the substrate 40 and/or stamp 50 and/or the sample 20, especially the elevations 58 of the stamp 50. Hence, with two or more lens configurations, one may select the a configuration to align the substrate 40, a configuration to align the stamp 50, a configuration to contact the elevations with the sample material 20 on the substrate 40.

FIG. 5c schematically depicts another type of substrate that may be used and that allows another method of “replication”. FIG. 5c schematically depicts a master substrate 4000, with substrate 40, wherein in the deepenings 158 of substrate 40 sample material 20 may be present. Substrate 40 is schematically depicted as raster. For further details of the substrate 40, see also above. The master substrate 4000 may further comprise a plurality of second substrates 410, indicated with references 410a-h. Such master substrate 4000 may for instance be an anopore plate, with above mentioned number of deepenings. The second substrates 410 and substrate 40 may be adjacent, but may also be arranged at a distance of each other, as in the schematic drawing 5c. After contacting the elevations 58 of stamp 50 with the sample material 20 in the deepenings 158 of the substrate 40, the stamp 50 with sample material 200, may subsequently be contacted with one or more of the second substrate 410. An advantage of using a master substrate 4000 with the substrate 40 (“mother”) and the plurality of second substrates 410 is that the master substrate 4000 may substantially be aligned once, whereas in the embodiment schematically depicted in FIG. 5b, each second substrate 410 may have to be aligned independently. For instance, a stamp 50 with 10000 elevations 58 and a master substrate 4000, with 100,000 deepenings 158 (belonging to substrate 40 and second substrate 410) may be applied, wherein the stamp 50 first contacts the sample material 20 in substrate 40, and then subsequently contacts the second substrates 410.

Contacting one or more elevations 58 of the stamp 50 with the sample material 200 (for instance from substrate 40) or replicating the sample material 200 on the one or more elevations 58 with a substrate (such as another part of substrate 40 or second substrate 410) may in an embodiment be performed by arranging the stamp 50 over the target (sample material or substrate), for instance at a distance L6 of about 0.2-250 μm, like about 2-20 μm, and then drop (especially by gravity) the stamp 50. This may be achieved by allowing the adaptor 100 release the stamp 50 (for instance by reducing or switching off vacuum or magnetic force). Thereafter, the stamp 50 may be retrieved again by the adaptor 100 for further processing, like replicating. Hence, in an embodiment, part of the distance L6 may be bridged by reducing L1, and part of L6 may be reduced (until contact), by releasing the stamp 50 from the adaptor.

Again, replicating one or more samples 200 may be controlled by inspection through the optical microscope 1000. This may (see also above) especially indicate that aligning the stamp 50 and another part of substrate 40 or second substrate 410, especially in a male-female configuration, and/or contacting the elevations with the sample material 20, is controlled by inspection through the microscope 1000. See further above for the explanation of “controlled by inspection through the optical microscope 1000”.

Hence, in this way a method for microscope delivery is provided; further, also a microscope, arranged to allow a (controlled) microscope delivery, is provided.

### EXAMPLES

The stamping apparatus is built around an Olympus microscope type BX41, equipped with a 10× eyepiece type WH10×, a 10× objective, a Marzhauser XY motorized XY table, a Z-axis motor, a Corvus controller and software. In addition the XY table is equipped with an extra home-made rotation facility necessary for the alignment procedure during the actual stamping process. The 10× objective is equipped with a home-made adapter to pick up and hold the stamp with vacuum. The microscope 1000 is also equipped with a CCD camera and software to view and capture pictures of the stamping process.

The stamping procedure is basically as follows:

1) Start and initialize microscope, Corvus controller, PC and Corvus software;

2) Place stamp 50 and anopore substrate 40 on substrate holder 42 (arranged to host substrate 40 and stamp 50);

3) Align the stamp 50 in the X-direction with the X-movement of the XY table using the home-made rotation facility;

4) Align the anopore substrate 40 to the X-direction of the XY table using the 3-points alignment option of the Corvus software;

5) Pick up the stamp 50 from the substrate holder 42 using the vacuum facility in the home-made adapter 100;

6) Pick up the sample 20 out of the deepenings 58 on the anopore substrate 40;

7) Move to the target location on the anopore substrates 410 (second substrates) and release the sample 200 in the target deepenings 158 on the anopore substrate 410.

### Experiment: Printing Bacteria Using a Replicator

L. plantarum WCFS1 was labelled with wheat germ agglutinin conjugated quantum dots (WGA:QDOTS, from Invitrogen, NL) to allow detection of the bacteria by fluorescence microscopy. After labelling, the QDOTS not binding to the bacteria were removed using a spun filter (0.2 micron, Millipore). A paste of this preparation was made containing labelled bacteria, 10% Ficoll (Sigma, NL) and 10% glycerol (Sigma, NL). The cellular paste was spread on a 36×8 mm strip of porous aluminium oxide (PAO) (Whatman, NL) at one end (an area of 8×8 mm, cell density>10e8/ml).

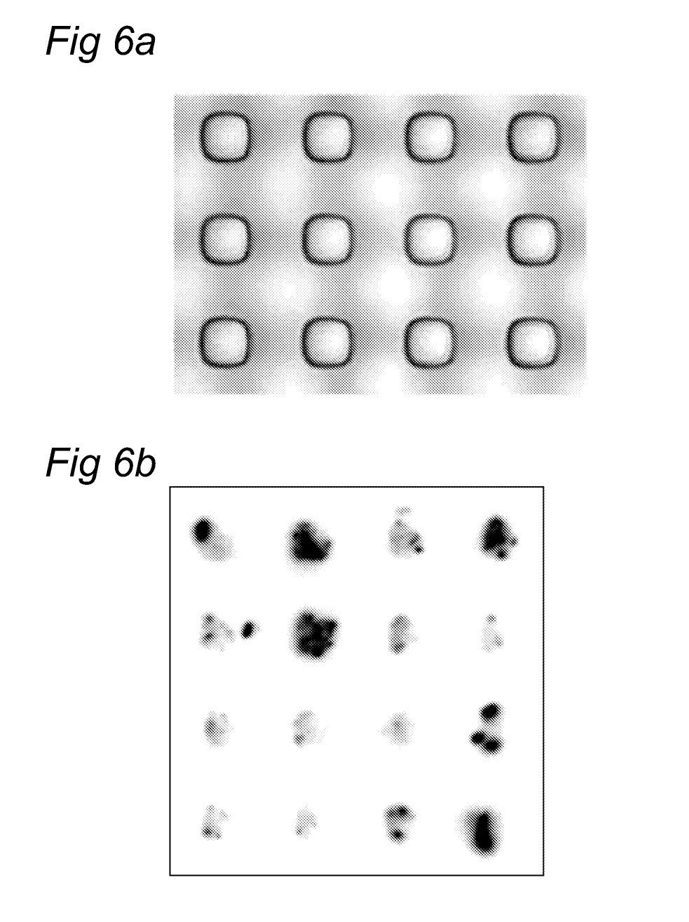

Printing with a microscope mounted replicator was as herein described, using pins (elevations) of 5×5 micron area (i.e. square tops 59) and 15 micron high (H1)) with 10 microns between distance between edges of directly adjacent pins. The replicator pins used are shown in FIG. 6a (this is part of the stamp having 400*400 pins). Loading with bacteria was by dropping the replicator (releasing it from the objective lens used to target the device) when the pins were 5 microns above the surface of the PAO covered with bacteria. In this way, the pins (elevations) were contacted with the sample material, thereby providing the pins with samples. Contacting the elevations with the sample material was controlled by inspection through the optical microscope.

Printing was by moving the replicator to a fresh area of PAO (without bacteria) and again dropping the replicator from the same height. In this way, the samples were subjected to a replication process.

After removal of the replicator the success in printing was assessed by fluorescence microscopy (see FIG. 6b, showing a detail of the printed area; individual bacteria and/or aggregates of bacteria are visible). For the first printing >99% success (at least one bacterium deposited per pin) was achieved. Subsequent outgrowth experiments suggested printed bacteria formed viable micro colonies by transfer of the PAO strips to MRS agar and incubation for 5 h followed by microscopy.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A method for taking a plurality of samples from a substrate with sample material by using an optical microscope and a stamp, wherein
the optical microscope comprises an objective with a lens, and a substrate holder, wherein a lens-holder distance (L1) between the lens and the substrate holder is variable, and
the stamp comprises at a side a plurality of elevations and wherein at least part of the stamp is transparent,
wherein the method comprises:
arranging at least part of the transparent part of the stamp in front of the objective, with the plurality of elevations directed to the substrate holder;
arranging the substrate with sample material to the substrate holder;
contacting the elevations with the sample material, thereby providing the plurality of elevations with samples, wherein contacting the elevations with the sample material is controlled by inspection through the optical microscope; and
subjecting the samples to a process selected from the group consisting of analysis and replication.

2. The method according to claim 1, wherein the sample material is selected from the group consisting of bacteria, virus, fungi, yeast and any other living cell or tissue.

3. The method according to claim 1, wherein the substrate comprises a k*l substrate deepenings array of deepenings with sample material, wherein k and l are independently in the range of 2-10,000.

4. The method according to claim 1, wherein the stamp comprises a n*m stamp elevations array of elevations, wherein n and m are independently in the range of 2-10,000, wherein neighbouring elevations are arranged at a shortest elevation distance (L2) in the range of 1-1000 μm, wherein the elevation height (H1) of the elevations is in the range of 0.1-1000 μm.

5. The method according to claim 4, wherein the array of the substrate and the stamp are alignable in a male-female configuration.

6. The method according to claim 1, wherein the method further comprises aligning the substrate and the stamp before contacting the elevations with the sample material.

7. The method according to claim 1, wherein the elevations of the stamp have tops, wherein the tops form an elevations top plane, wherein the microscope allows, without changing the objective, a first lens configuration and a second lens configuration, wherein the first lens configuration allows a sharp image of at least part of the elevations top plane and wherein the second lens configuration allows a sharp image of at least part of the substrate at a distance between the substrate and the tops larger than 0 cm.

8. The method according to claim 8, wherein the first or the second lens configuration is selectable by introducing a second lens between lens and an ocular.

9. The method according to claim 1, further comprising:
transporting the stamp with samples to an analysis unit for analysis of one or more of the samples.

10. The method according to claim 1, further comprising:
contacting the elevations with samples with one or more second substrates.

11. The method according to claim 1, further comprising:
contacting the elevations with samples with another part of the substrate than from the part where the sample material was retrieved from.

12. The method according to claim 1, wherein the plurality of elevations comprise elevations which are provided with one or more auxiliary compounds selected of the group consisting of acids, bases, antibodies, organic catalysts, inorganic catalysts, markers, receptor ligands, oligonucleotides, and DNA-binding proteins.

13. A stamp comprising at a side a plurality of elevations, wherein the stamp comprises an n*m stamp elevations array of elevations, wherein n and m are independently in the range of 2-10,000, wherein neighbouring elevations are arranged at a shortest elevation distance (L2) in the range of 1-1000 μm, wherein neighbouring elevations, wherein the elevation height (H1) of the elevations is in the range of 0.1-1000 μm, and wherein at least part of the stamp is transparent.

14. The stamp according to claim 13, wherein the stamp substantially consists of a transparent material selected from the group consisting of PMA (polymethacrylate), PMMA (polymethylmethacrylate), PC (polycarbonate), PDMS (polydimethyl siloxane), perspex, quartz, gemstones, glass, ceramic materials.

15. An optical microscope comprising an objective with a lens, an adaptor, and a substrate holder for a substrate, and a stamp, wherein the adaptor is arranged to receive a stamp comprising at a side a plurality of elevations, wherein the stamp comprises an n*m stamp elevations array of elevations, wherein n and m are independently in the range of 2-10,000, wherein neighbouring elevations are arranged at a shortest elevation distance (L2) in the range of 1-1000 μm, wherein the elevation height (H1) of the elevations is in the range of 0.1-1000 μm, and wherein at least part of the stamp is transparent, wherein the adaptor is further arranged to align at least part of the transparent part of the stamp in front of the objective.

16. An optical microscope, according to claim 15, wherein the adaptor further comprises one or more seizing devices, wherein the one or more seizing device are arranged to attach the stamp to the adaptor, and wherein the one or more seizing devices are selected from the group consisting of a vacuum channel, connectable to a vacuum pump, with opening, an (electro)magnet and an electrostatic device.

17. The optical microscope according to claim 15, wherein the optical microscope is a microscope for visible light.

18. The optical microscope according to claim 15, wherein the substrate and the stamp are rotatable in parallel planes.
